(19) United States
(12) Patent Application Publication
(10) Pub. No.: US 2006/0047217 A1
(43) Pub. Date: Mar. 2, 2006

(54) Method and Apparatus for Infant Sleep Apnea Monitoring and Data Analysis

(76) Inventors: Mohsen Mirtalebi, Indianapolis, IN (US); Andrea Pankiewiuz Mirtalebi, Indianpolis, IN (US)

Correspondence Address: MOHSEN MIRTALEBI, 1919 HOYT AVENUE, INDIANAPOLIS, IN, 46203, US

(21) Appl. No.: 11/161,519

(22) Filed: Aug. 6, 2005

Related U.S. Application Data
(60) Provisional application No. 60/522,046, filed on Aug. 6, 2004.

(51) Int. Cl. A61B 5/08; A61B 5/00

(52) U.S. Cl. 600/534; 600/549

(57) ABSTRACT

Methods and apparatuses for monitoring breathing pattern by monitoring and analyzing data that are the results of pressure changes in an air bed due to amount of air inhaled and exhaled and the chest expansion and contraction of an infant. The changes in pressure over time can be recorder, modeled and sent to be analyzed for fault detection in breathing pattern. As a result the abnormalities in the breathing pattern can be detected and parents and pediatrics can be warned in case of an Apparent Life-Threatening Event (ALTE) to prevent Sudden Infant Death Syndrome (SIDS).

### FIELD OF THE INVENTION

The present invention relates to methods and apparatus for monitoring respiration and related data analysis to determine whether there is an abnormality in the breathing pattern of Infant using mathematical modeling techniques.

### BACKGROUND OF THE INVENTION

It is the instinct fear of parents that causes them to tiptoe to their babies' room in the middle of night to make sure that their babies' tiny chests are still moving. The fear of sudden infant death syndrome (SIDS) is real. It costs lives of 5,000 to 7,000 infants between the ages one month to one year each year in the United States alone. The fear is real but the cause is not known yet.

Most experts in the U.S. believe that there is a strong relationship between sleep apnea and SIDS but this relationship has not been clearly identified.

Sleep apnea in the infants with the duration of 5 to 8 seconds is completely normal. If baby moves around a lot then a pause of 10 to 15 seconds is also normal. A prolonged apnea that lasts more than 20 seconds is considered Apparent Life-Threatening Event (ALTE).

Babies can be saved if an ALTE is detected quickly enough. There are different responses to awaken the baby from an apnea. They range from a mild stimulation such as flicking infant's finger to mouth and nose resuscitation depending on how fast parents respond to the first sign of ALTE. In addition to a horrifying death as a result of undetected ALTE, a late response can result in infant's permanent brain damage.

There are two common categories for infant apnea. Category 1 is named Central Apnea, in which the baby makes no effort to breath. Category 2 is named Obstructive Apnea, in which the baby has chest movement but there is no air flow though the mouth and nose to the lung.

For each category of infant apnea there are corresponding sleep apnea monitors currently on the market. Group 1 detects infant's chest movement for monitoring Central Apnea. Group 2 in addition to detecting chest movement, monitors other physiological functions such as heart rate and brain activities (group 2).

There are some disadvantages associated with each group of currently available monitors. For the group 1, the monitor cannot detect Obstructive Apnea because there is chest movement involved with this type of apnea. The group 2 of monitors is hard to operate by parents and there are frequent false alarms that can be caused based on non-apnea grounds such as, loosened wire connections and shallow breathing due to infant's abdominal breathing. There is also another problem that is common between the two groups which is that, as baby grows the duration of normal pauses become longer then this will cause more false alarms until a health professional readjusts the monitor.

### SUMMARY OF THE INVENTION

Since a few seconds can make a difference between life and death, this invention can forecast an ALTE a few seconds ahead of time by observing, recording, and mathematically modeling infant's breathing pattern.

The present invention is utilizing an air bed made of polyester and nylon materials that equipped with pressure sensor along with built-in microprocessors that can communicate with a remote computer to do the monitoring and data analysis along issuing warning and alarms.

The pressure transducer and temperature sensor are the ones that are currently available in the market. The pressure sensor that is temperature compensated, calibrated, and amplified, acquires data that is generated by the infant's chest movement to the built-in microprocessor.

The microprocessor records the sensed pressure and keeps the data in an array of numbers. Then the microprocessor will start to analyze the data to find out the most appropriate mathematical model using statistical process methods such as Auto Regressive Moving Average (ARMA/ARX) models or Exponential Smoothing methods. The model's parameters will be sent to the off-site computer through a modem and a transmission line. Data then will be classified using data mining techniques.

The data transmission from infant's bed establishes a stream of data to the off-site computer, which always reconstructs and saves the model in its hard-drive memory. Then the same computer compares the saved data against a stream of incoming data from infant's bed and starts to analyze the data to come up with that specific model's class characteristics.

After the model is processed, adjustments in calculations are placed and the errors in the breathing pattern are identified, the off-site computer begins to forecast infant's breathing pattern and possibly notify the infant's bed of an upcoming ALTE. Then infant's bed triggers a range of alarms depending the severity of the warning.

In addition, in case of down time in the transmission line, a stand-alone alarm circuitry is provided with the bed to detect chest movement and the heart rate of the baby.

Also, any faulty operation in the bed's circuitry and/or its physical behavior such as bed deflation can be detected in real time and parents can be notified immediately.

Finally, any necessary adjustment and software maintenance that is needed because of changes in the breathing pattern due to the infant's growth can be applied on-line.

### DISCRIPTION OF DRAWINGS

FIG. 1 is an illustration of the air bed in the infant's crib.

FIG. 2 is a diagram which shows the location of alarm and control circuitry in the bed.

FIG. 3 shows the main electronic components and sensors that will be used to achieve the scope of the device for monitoring and control purposes.

FIG. 4 illustrates the dynamic of infant's chest movement and its interaction with the bed.

FIG. 5 shows the operating flowchart of the whole system including on-site monitoring devices, off-site computer and a suggested method of monitoring and alarm system.

FIG. 6 shows a possible normal breathing pattern recorded by the bed and the error curve with respect to an ideal fitted curve.

FIG. 7 shows a possible abnormal breathing pattern that might be recorded by the bed and a method for detecting error and abnormalities in the breathing pattern.

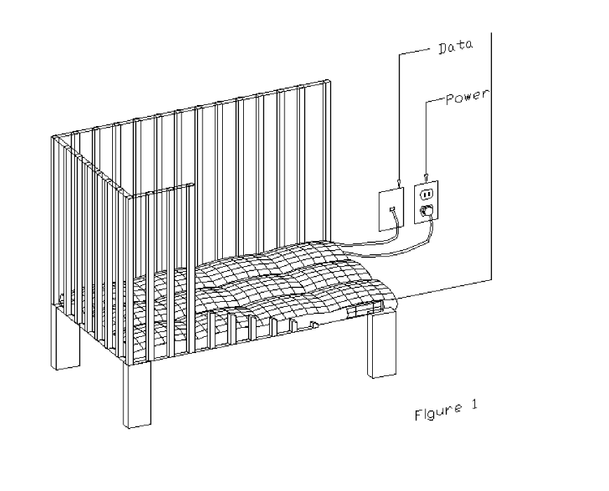

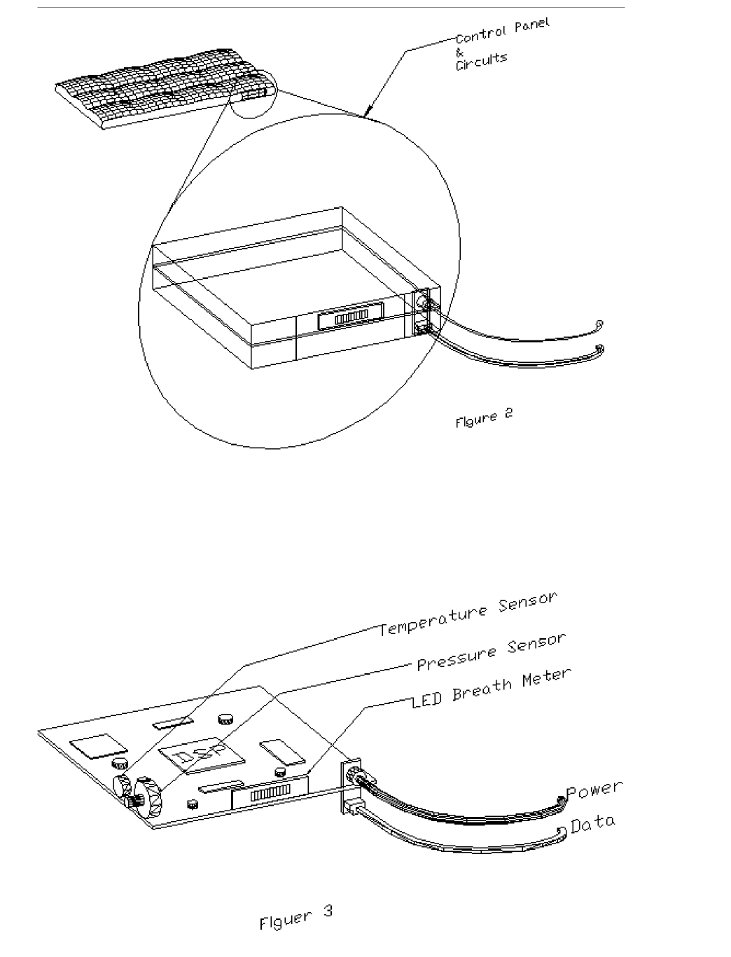

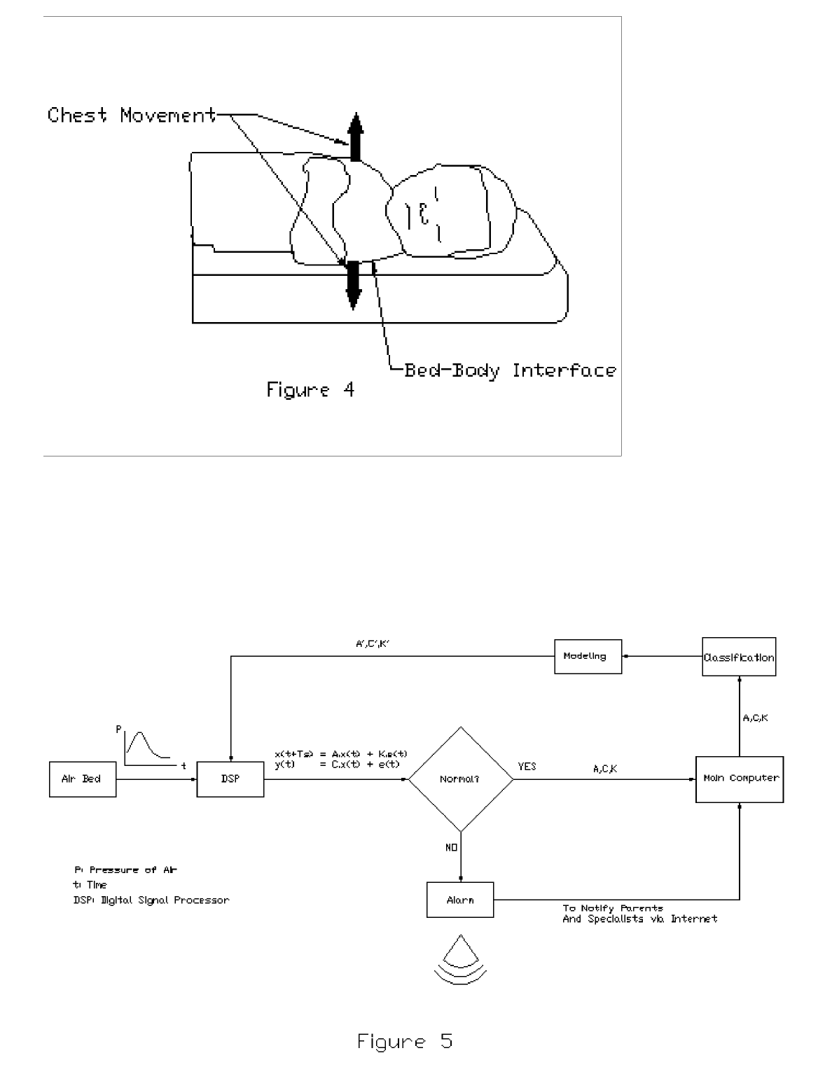

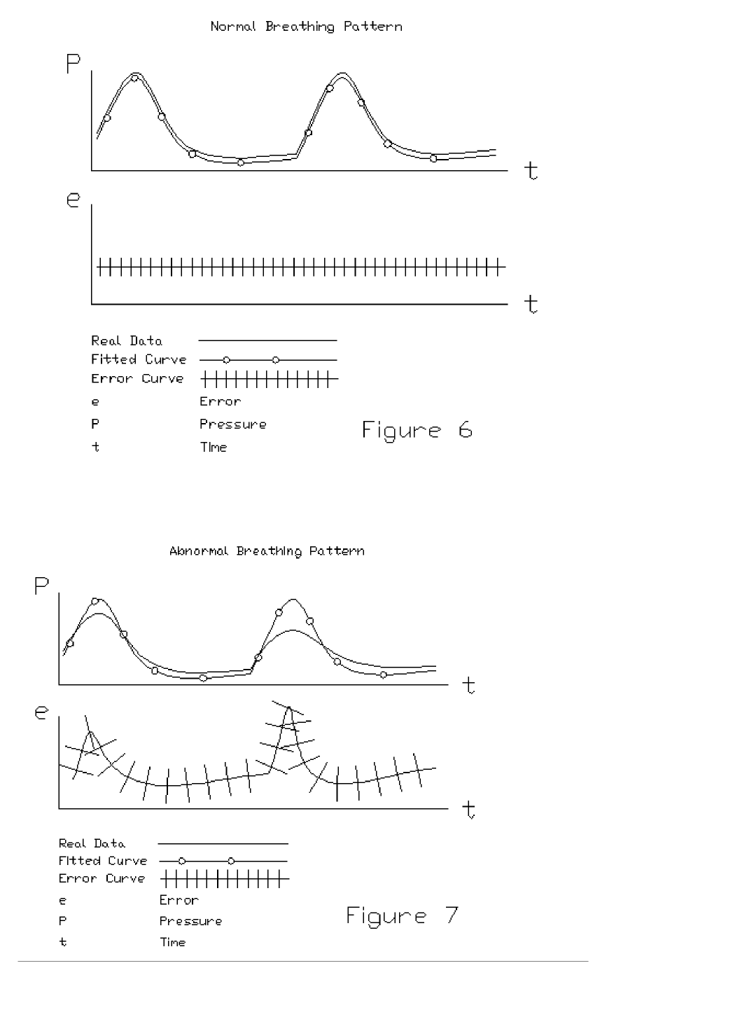

### TECHNICAL FIELD

The followings are the description of the technical field.

### DETAILD DESCRIPTION OF THE INVENTION

The present invention generally relates to an assay for detecting abnormalities in infant's breathing pattern, triggering alarm, classifying various breathing patterns, modeling infant's breathing pattern and forecasting an ALTE to prevent SIDS.

Since the current instruments in the market for monitoring breathing pattern are either expensive and complicated or inexpensive and dysfunctional, there is a need for an instrument that is simple, accurate, and inexpensive.

This system has a user friendly interface and many features including an on-line communication channel that enables it to operate automatically and independently for the most part.

As soon as the bed is plugged into the power outlet and a phone line, it starts to gather vital data of its surrounding through its temperature and pressure transducers.

With the presence of the infant, the pressure and temperature sensors on the airbed will gather the vital information in small portions. The microprocessor will choose some portions of the streaming data for data analysis purposes. These series of data packages then will be modeled. The data pack is typically a function of pressure and time. This function will be carefully analyzed for modeling purposes. The microprocessor will start to find a primary and ordinary model of the function with state-space representation such as x(t+Ts)=A.x(t)+K.e(t) and y(t)=C.x(t)+e(t) in which A, K, and C are system parameters, t is time variable and Ts is time interval. Since the input to the system is not known the system will be treated with time series analysis without input.

The microprocessor will acquire A, C, and K parameters and transfers the data to the off-site computer for final analysis. These data along with hundreds of other data stream from other beds will be analyzed and classified with data mining techniques. Each class of data then will have a class indicator with A′, C′, and K′ prime parameters.

The prime parameters will be soon sent back to each bed that is the member of the same class. Then the error analysis will be performed and a model will be made with A, C, and K parameters. These data will get through the same process that was explained in the previous paragraphs.

In the meantime the bed is sensing the infant's body along with the climate temperature to monitor the baby's presence and to correct the pressure calculations.

The on-site alarm system will be activated in two cases. One is when it does not detect any movement for 20 seconds or will be activated when the bed forecasts an ALTE.

What is claimed is:

1. In an apparatus as an intelligent bed with built-in microprocessor, pressure and temperature sensors, stand alone and battery operated alarm circuitry, communication circuitry for communicating with the main computer in claim 2, and computer software that implement the monitoring and fault detection algorithm in claim 3.

2. In an apparatus as a network of remote computers located in sleep apnea centers with the ability of monitoring high number of beds, calculating complex mathematical algorithm in claim 3, analyzing incoming stream of data in real time, communicating with every bed in claim 1 and setting bed's alarm and issuing warnings appropriately.

3. In a method for monitoring infants breathing pattern that is acquired by the bed in claim 1 using statistical process control and monitoring and classification methods utilizing a network of computers in claim 2 to find whether there is any error in the breathing pattern and whether to notify caregivers.

4. In a method for finding the most appropriate and unique model that describes the data received from the air bed in claim 1 that concludes the breathing pattern for each unique individual infants using statistical process control techniques in claim 3 such as Triple Exponential Smoothing.

5. In a method for advanced data analysis, pattern recognition, and data classification by the computers in the remote sleep apnea centers in claim 2, to collect data from different beds in claim 1 at different geographical areas comprising.

6. In a method for classifying all breathing patterns through data mining techniques in claim 3.

7. In a method for finding abnormalities and forecasting each individual infant's breathing pattern in claim 3 based on the class they belong to.
